(19) United States
(12) Patent Application Publication
(10) Pub. No.: US 2023/0101998 A1
(43) Pub. Date: Mar. 30, 2023

(54) MODELING AND VISUALIZING ST SEGMENT MORPHOLOGY FOR DISCRIMINATING STEMI FROM CON-FOUNDERS

(71) Applicant: KONINKLIJKE PHILIPS N.V., EINDHOVEN (NL)

(72) Inventors: REZA FIROOZABADI, THOUSAND OAKS, CA (US); SAEED BABAEIZADEH, ARLINGTON, MA (US); RICHARD EARL GREGG, WESTFORD, MA (US)

(21) Appl. No.: 17/908,616

(22) PCT Filed: Mar. 5, 2021
(86) PCT No.: PCT/EP2021/055531

§ 371 Date: Sep. 1, 2022

Related U.S. Application Data
(60) Provisional application No. 62/985,600, filed on Mar. 5, 2020.

(51) Int. Cl. A61B 5/24; A61B 5/00

(52) U.S. Cl. CPC A61B 5/24; A61B 5/7203

(57) ABSTRACT

A system and method for modeling and visualizing ST segment morphology in an ECG. Many cardiac conditions show ST-elevation in ECG data and may be misdiagnosed as a consequence. The exemplary embodiments model a segment in the ECG with a curve and extract features from the curve to discriminate between the cardiac conditions, including STEMI.

### BACKGROUND

An electrocardiogram (ECG or EKG) may be used to diagnose a cardiac condition, including ST-segment elevation myocardial infarction (STEMI), a form of heart attack. ECGs are produced by recording the heart's electrical activity via two or more electrodes affixed to the skin and arranged at different anatomical locations around the heart. The ECG is output as one or more graphs of voltage over time, the voltage corresponding to the electrical potential difference between two of the electrodes. A pair of electrodes forms a “lead,” and a typical ECG is a 12-lead ECG, each lead measuring the heart's electrical activity from a different angle.

A typical ECG 300, as shown in FIG. 3, is characterized by electrical activity following a pattern including portions known as the P wave 302 (representing the depolarization of the atria), the QRS complex 304 (representing the depolarization of the ventricles), the T wave 306 (representing the repolarization of the ventricles) and the U wave 308 (theorized to represent “afterdepolarizations” in the ventricles, but the origin of which is not definitively known). The QRS complex 304 ends at the J-point 310, with the ST segment 312 connecting the QRS complex and the T wave 306. The ST segment represents the period between depolarization and repolarization of the ventricles. It may be difficult at times to determine exactly where the ST segment 312 ends and the T wave 306 begins, so the JTpeak interval 314, beginning at the J-point 310 and ending at the apex of the T wave 306, may be considered when interpreting ECG data.

An elevated ST segment may be indicative of a number of cardiac conditions including pericarditis disease, early repolarization syndrome, left bundle branch block (LBBB), left ventricular hypertrophy (LVH), and STEMI. STEMI is diagnosed by criteria identifying the ST levels beyond the normal thresholds in at least two contiguous leads in a multi-lead, e.g. 12-lead, ECG set. However, misdiagnosis of STEMI as one of its confounding conditions, or misdiagnosis of a confounding condition as STEMI, may occur, especially when treating patients presenting atypical symptoms. A STEMI confounder is a cardiac condition showing similar ST-elevation as STEMI, making a diagnosis of the actual disease difficult.

The absolute amplitude of an ST segment deviation and/or the extent of the ST segment deviation, as reflected in the number of leads with threshold ST elevation, is commonly correlated with the size and/or severity of the ischemia, i.e. the restriction of blood supply. However, the correlation has been found to be weak in some cases, and the ST segment deviation may be attenuated when opposing segments of the heart show concomitant ischemia. Misinterpretation of ST-elevation originated by STEMI confounders such as pericarditis and early repolarization may cause unnecessary medical treatments and result in extra costs to the patients, responders, and medical centers. Misdiagnosis of actual STEMI as other confounders, on the other hand, may cause delay in treatment of the STEMI patient and put the patient's health at risk with detrimental consequences.

### SUMMARY

The present disclosure is directed to a method including identifying a segment in ECG data, modeling the segment with a curve, extracting features from the curve, determining a concavity of the segment based on the extracted features and determining a cardiological condition based on the concavity of the segment.

The present disclosure is further directed to a system including a memory configured to store ECG data. The system includes a processor configured to perform operations comprising identifying a segment in the ECG data, modeling the segment with a curve, extracting features from the curve, determining a concavity of the segment based on the extracted features and determining a cardiological condition based on the concavity of the segment.

### BRIEF DESCRIPTION OF THE DRAWINGS

FIG. 1 shows a system for modeling and visualizing the ST segment morphology in ECG data and distinguishing STEMI from its confounding conditions based on the model.

FIG. 2 shows a method for modeling and visualizing the ST segment in ECG data and distinguishing STEMI from its confounding conditions based on the model.

FIG. 3 shows an exemplary ECG.

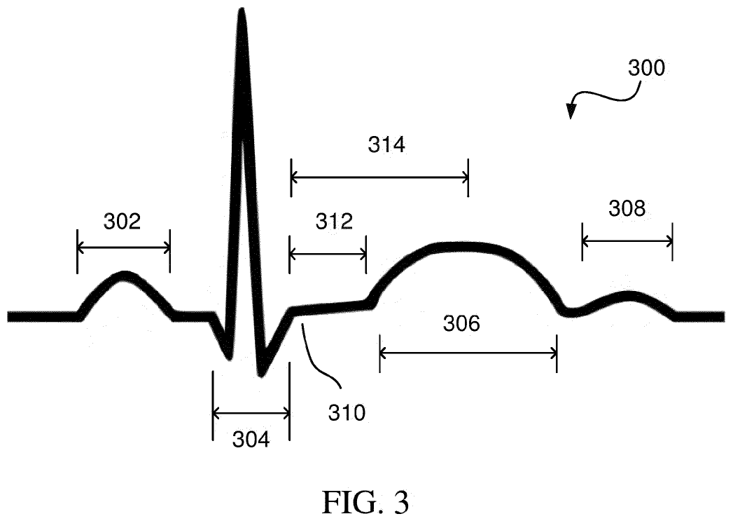

FIG. 4 shows an exemplary representative beat for an ECG lead, with various data analyses applied thereon.

FIG. 5a shows a first set of 12-lead ECG representative beats for a pericarditis condition.

FIG. 5b shows the magnified JTpeak intervals for the representative beats of FIG. 5a.

FIG. 6a shows a second set of 12-lead ECG representative beats for an early polarization condition.

FIG. 6b shows the magnified JTpeak intervals for the representative beats of FIG. 6a.

FIG. 7a shows a third set of 12-lead ECG representative beats for a STEMI condition.

FIG. 7b shows the magnified JTpeak intervals for the representative beats of FIG. 7a.

FIG. 8a shows a fourth set of 12-lead ECG representative beats with an osculating circle visualized thereon.

FIG. 8b shows the fourth set of representative beats with minimum curvature radius and maximum curvature radius printed thereon.

### DETAILED DESCRIPTION

The exemplary embodiments may be further understood with reference to the following description and the appended drawings, wherein like elements are referred to with the same reference numerals. The exemplary embodiments relate to systems and methods for modeling and visualizing the morphology of the ST segment of an ECG for distinguishing an ECG indicating STEMI from an ECG indicating one of its confounding conditions showing similar ST elevation as STEMI.

The exemplary embodiments will be described with reference to criteria for distinguishing STEMI from its confounders, however, it should be understood that the exemplary embodiments are not limited thereto. For example, other cardiac conditions, or even non-cardiac conditions, may affect an ECG in such a way that modeling the JTpeak interval may provide information that can, when compared to condition-specific criteria, distinguish between the condition and conditions showing similar ECG data. Additionally, segments of the ECG other than the JTpeak interval may be modeled and information derived from the morphology.

The morphology of the ST segment (e.g., concave upward, concave downward, or straight line) is one of the ECG features which, along with other ECG features specific to each disease, may discriminate STEMI from its confounders. For example, concave downward (upward convex) or straight-line ST segments are not typically present in pericarditis or early repolarization. Thus, a lack of upward concavity in the ST segment is a feature that may be used to distinguish between STEMI and these other conditions. Concave upward (or convex downward) is defined herein as a curve having a positive second derivative, while concave downward (or convex upward) is defined herein as a curve having a negative second derivative. Some curves fitted to ECG data may have so much noise that, although the best-fit curve is concave upward, the data is not considered concave upward for the purposes of the exemplary embodiments.

In prior methods, the concavity of the ST segment is determined by modeling a line between the J-point to the apex of the T wave and determining the point in the JTpeak interval with a maximum distance from the line (below or above the line), or determining the sign of the area between the line and the JTpeak interval. However, these methods could be inaccurate in the presence of artifacts. Artifacts are very common in ECG data, and may be caused by, e.g., muscle artifact, baseline wander and powerline noise.

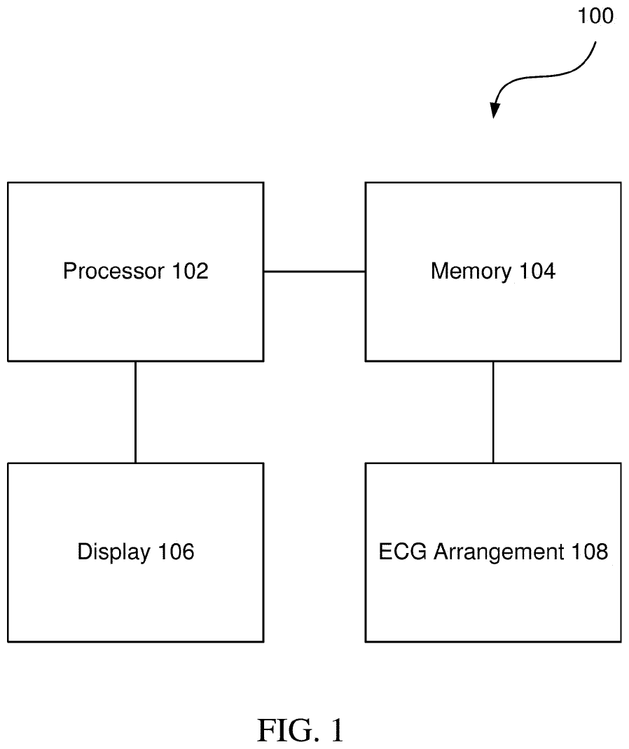

FIG. 1 shows a system 100 for modeling and visualizing the ST segment morphology and distinguishing STEMI from its confounding conditions based on the model. The system 100 includes a processor 102 for analyzing ECG data stored on a memory 104 and fitting a curve to the ECG data. The memory 104 may be coupled to an ECG arrangement 108, the ECG arrangement 108 comprising e.g. the electrodes forming the 12 leads, the wiring extending therefrom, one or more amplifiers, one or more processing arrangements, etc. for producing the ECG graphs and storing the graphs to the memory 104 in substantially real time. Thus, the exemplary embodiments are intended for rapid diagnosis of an ongoing cardiac condition. The processing arrangement(s) of the ECG arrangement 108 may, in some embodiments, encompass the processor 102 for performing the exemplary calculations. Thus, the processing of the data from the ECG arrangement 108 and the generation of the curves and associated features may be implemented in separate modules of a single processor. Alternately, the processor 102 may be a separate component and the memory 106 may store previously acquired ECG data so that the exemplary embodiments may be performed on previously acquired (i.e., not real-time) data.

The processor 102 additionally extracts mathematical and geometrical features from the curve fitted to the ECG data. For example, a concavity of the curve is assessed, and it is determined whether the ST segment shape (as best-fit modeled by the processor 102) satisfies a given condition, to be explained in greater detail below. A display 106 is configured to visualize the curve and the related mathematical/geometrical features.

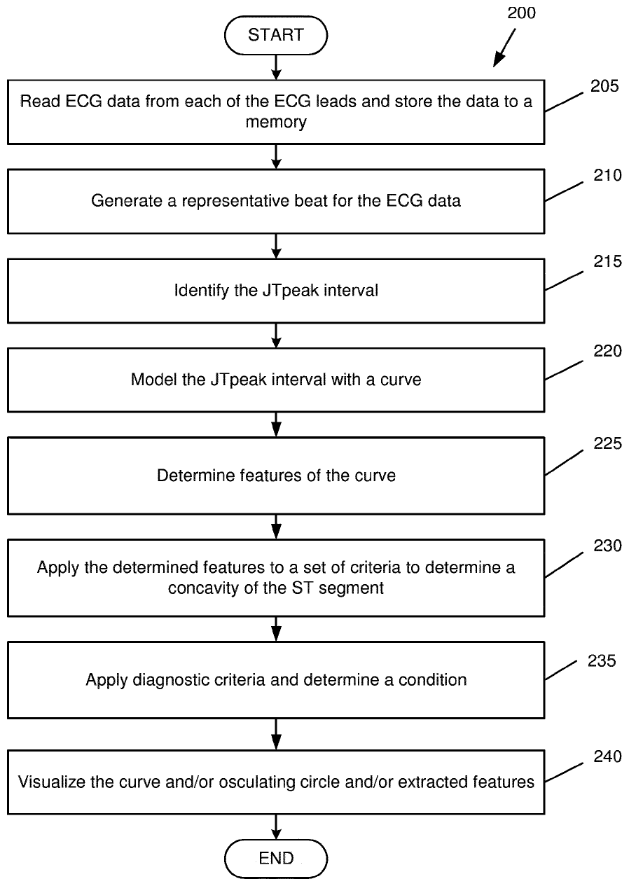

FIG. 2 shows a method 200 for modeling and visualizing the ST segment morphology in ECG data and distinguishing STEMI from its confounding conditions based on the model. The exemplary embodiments will be described with respect to a typical 12-lead ECG, however, the features described herein are not limited thereto.

In 205, ECG data is read from each of the ECG leads and a graphical representation, i.e., the ECG, is generated for each of the leads and stored to the memory 104. In the following description, each of the steps 205-230 is performed for each of the ECG leads (e.g. 12 times for a 12-lead ECG). Thus, the morphology of the ECG is assessed for each of the leads.

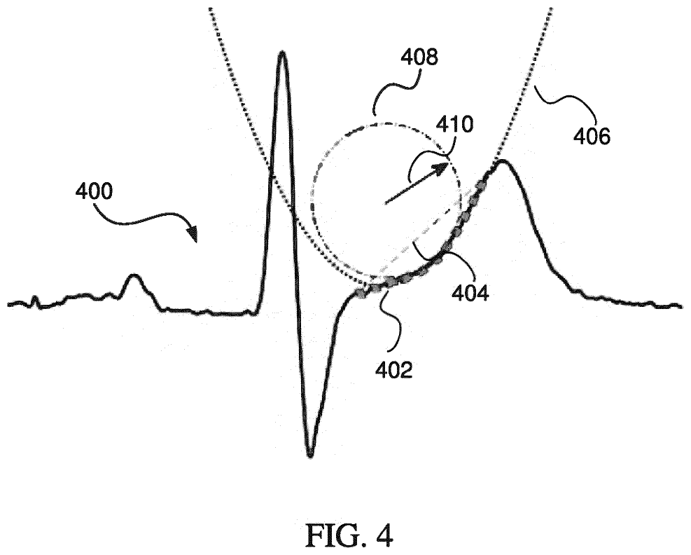

In 210, a representative beat is generated for the ECG data. The representative beat is representative of one heartbeat cycle, i.e., the progression through the full atrial and ventricular depolarization and repolarization of the heart. An ECG typically gathers data for at least ten seconds, thus, the data may encompass multiple heartbeat cycles. In the exemplary embodiments described herein 10 seconds of data is analyzed, however, a longer interval such as 30 seconds may be used to exclude any potential artifacts and give a cleaner average. The representative beat may be generated by, e.g., averaging the multiple heartbeats, with noisy or abnormal beats being removed. FIG. 4 shows an exemplary representative beat 400 for an ECG lead, with various data analyses applied thereon, to be explained in further detail below.

In 215, the ST segment morphology is identified for the representative beat. The beginning and the end of the JTpeak interval (i.e. the fiducial points) are determined. As discussed previously, the JTpeak interval begins at the J-point and ends at the apex of the T wave, which may be detected in the data using known methods. FIG. 4 shows the JTpeak interval 402 as a curved dotted line, with a straight line 404 connecting the boundary points of the JTpeak interval 402.

In 220, the identified JTpeak interval is modeled with a curve. In the exemplary embodiments described herein a second-order curve (e.g. a parabola) is fit to the JTpeak interval. However, the JTpeak interval may be modeled with a curve in unlimited ways, e.g., with a higher-order polynomial or with a second-order model other than that described below.

The exemplary quadratic polynomial regression approach described herein fits a section of a parabola to the JTpeak interval identified in the ECG data. The coefficients a, b, and c are the parameters to determine:

ŷ=a+bx+cx²

The exemplary approach described herein estimates the polynomial coefficients in a fast-analytic way. In a JTpeak interval comprising N samples: (xₖ,yₖ), k=1, . . . ,N, the sum of the squared error between the fitted curve (ŷ) and the original signal (y) at each sample k is minimized using least squares estimation:

$${e}_{k} = {y}_{k} {\hat{y}}_{k} = {y}_{k} - ( a + bx + {cx}^{2} ) \\ S = {\sum }_{k} {( {e}_{k} )}^{2} = {\sum }_{k} {( {y}_{k} - ( a + bx + {cx}^{2} ) )}^{2} \\ \frac{\partial S}{\partial a} = 0 , \frac{\partial S}{\partial b} = 0 , \frac{\partial S}{\partial c} = 0$$

The above partial derivatives lead to the following system of 3 equations:

$${\sum }_{k} y = a . N + b . {\sum }_{k} x + c . {\sum }_{k} {x}^{2} \\ {\sum }_{k} xy = a . {\sum }_{k} x + b . {\sum }_{k} {x}^{2} + c . {\sum }_{k} {x}^{3} \\ {\sum }_{k} {x}^{2} y = a . {\sum }_{k} {x}^{2} + b . {\sum }_{k} {x}^{3} + c . {\sum }_{k} {x}^{4}$$

The above system of 3 equations and 3 unknowns is solved using Cramer's rule:

$$a = \frac{{Δ}_{a}}{Δ} , b = \frac{{Δ}_{b}}{Δ} , c = \frac{{Δ}_{c}}{Δ} where : Δ = [ \begin{matrix}N & {\sum }_{k} x & {\sum }_{k} {x}^{2} \\ {\sum }_{k} x & {\sum }_{k} {x}^{2} & {\sum }_{k} {x}^{3} \\ {\sum }_{k} {x}^{2} & {\sum }_{k} {x}^{3} & {\sum }_{k} {x}^{4}\end{matrix} ] and \\ {Δ}_{a} = [ \begin{matrix}{\sum }_{k} y & {\sum }_{k} x & {\sum }_{k} {x}^{2} \\ {\sum }_{k} xy & {\sum }_{k} {x}^{2} & {\sum }_{k} {x}^{3} \\ {\sum }_{k} {x}^{2} y & {\sum }_{k} {x}^{3} & {\sum }_{k} {x}^{4}\end{matrix} ] , \\ {Δ}_{b} = [ \begin{matrix}N & {\sum }_{k} y & {\sum }_{k} {x}^{2} \\ {\sum }_{k} x & {\sum }_{k} xy & {\sum }_{k} {x}^{3} \\ {\sum }_{k} {x}^{2} & {\sum }_{k} {x}^{2} y & {\sum }_{k} {x}^{4}\end{matrix} ] , {Δ}_{c} = [ \begin{matrix}N & {\sum }_{k} x & {\sum }_{k} y \\ {\sum }_{k} x & {\sum }_{k} {x}^{2} & {\sum }_{k} xy \\ {\sum }_{k} {x}^{2} & {\sum }_{k} {x}^{3} & {\sum }_{k} {x}^{2} y\end{matrix} ]$$

These determinants are written in closed form as:

Δ=N·(m2·m4−m3·m3)−m1·(m1·m4−m3·m2)+m2·(m1·m3−m2·m2)

Δₐ=z1·(m2·m4−m3·m3)−m1·(z2·m4−z3·m2)+m2·(z2·m3−z3·m2)

Δ_{b}=N·(z2·m4−z3·m3)−z1·(m1·m4−m3·m2)+m2·(z2·z3−z3·z2)

Δ_{c}=N·(m2·z3−m3·z2)−m1·(m1·z3−m3·z2)+z1·(m1·m3−m2·m2)

where:

m1=Σₖx, m2=Σₖx², m3=Σₖx³, m4=Σₖx⁴z1=Σₖy, z2=Σₖxy, z3=Σₖx²y

By replacing the polynomial coefficients, the fitted curve may be written as:

ŷ=(Δₐ+Δ_{b}x+Δ_{c}x²)/Δ

An exemplary polynomial curve 406 fitted to the JTpeak interval 402 is shown in FIG. 4. An osculating circle 408, i.e., a circle having a curvature equal to a curvature of the polynomial curve 406 at a given point, may be determined as well. The osculating circle 408 shown here has a curvature equal to the curvature at the point on the curve 406 closest to the midpoint of the JTpeak interval, however, the osculating circle may be generated for any point on the curve 406. A radius 410 of the osculating circle 408 at a given point is an equivalent concept to the radius of curvature of the polynomial curve 406 at the given point. A maximum and/or minimum radius of curvature of the JTpeak interval may be used in the calculations for determining the nature of the cardiological condition, and/or may be displayed to a user manually interpreting the ECG data, to be described below.

It may be seen in FIG. 4 that the polynomial curve 406 fits the JTpeak interval 402 very well. However, in practice, ECG data may not be so smooth as the representative beat 400. Typically, there will be greater variation in the ECG data and there will be model error to account for.

In 225, the model error, and other features of the polynomial curve 406 are determined. In the exemplary embodiments described herein a model error, noise level, vertex location, and curvature (specifically a maximum curvature) are determined for use in determining the cardiological condition. The noise level, for example, is measured by comparing the fitted curve to the original ECG. However, different features may be defined and used for different applications.

Model error (R²) may be defined as:

$${R}^{2} = 1 - \frac{\sum {( \tilde{y} - \hat{y} )}^{2}}{\sum {( \tilde{y} - {\tilde{y}}_{ave} )}^{2}}$$

where \tilde{y} is the smoothed JTpeak interval using a moving average filter.

Noise level (err) may be defined as:

$$err = \frac{\sum \vert y - \tilde{y} \vert}{N . \vert {\tilde{y}}_{m a x} - {\tilde{y}}_{m i n} \vert}$$

Vertex location (v) may be defined as:

$$v = - \frac{b}{2 c}$$

Curvature (κ) may be defined as:

$$κ = \frac{\vert {\hat{y} ( x )}^{″} \vert}{{( 1 + {\hat{y} ( x )}^{{′}^{2}} )}^{3 / 2}}$$

with κₘ being defined as the maximum curvature during the JTpeak interval.

In 230, the determined features of the best-fit curve are applied to a set of criteria to determine the concavity of the ST segment. Various criteria may be defined for making the determination. In one example, the ST segment is considered upward concave if the following condition is met:

Cond=(c≥thr1) & (R²≥thr2) & (err<thr3) & (v<thr4) & (κₘₐₓ>thr5)

where thr1, thr2, thr3, thr4, and thr5 are the thresholds defined for performance of the concavity detection. Thus, it may be seen that an upward concavity determination is dependent upon the second order coefficient c, the model error R², the noise level err, the vertex location v, and the maximum curvature κₘₐₓ. If any one of the criteria are not met, the ST segment may be considered to be non-concave-upward. A determination of non-concave-upward may not be equivalent to a determination of concave downward. Rather, the determination takes into account not only the curve but the differences between the curve and the actual data, and signifies that the ST segment cannot be said to be definitively concave upward.

As mentioned previously, the method steps 205-230 are performed for each of the ECG leads. Thus, a determination of concave upward or non-concave-upward is made for the ST segment for each of the leads.

In 235, diagnostic criteria are applied in view of the aforementioned features and concavity determinations to determine a condition. In the exemplary embodiments described herein, STEMI is distinguished from pericarditis and early repolarization. However, as mentioned previously, other conditions may be assessed based on other features extracted from the ECG data.

Pericarditis is identified by widespread ST elevation in an upward concave ST segment and PQ-segment depression in some leads. Early repolarization is recognized by ST elevation in upward concave ST segment in some leads with no reciprocal ST depression in other leads, and T-waves or J-point notches and slurs, tall positive T-waves, and low ST/T ratios. As discussed previously, concave upward or straight-line (i.e., non-concave upward) ST segments are typically not present in pericarditis or early repolarization. Thus, a presence of a non-concave-upward curve in any of the e.g. 12 ECG leads may indicate STEMI as opposed to these confounders.

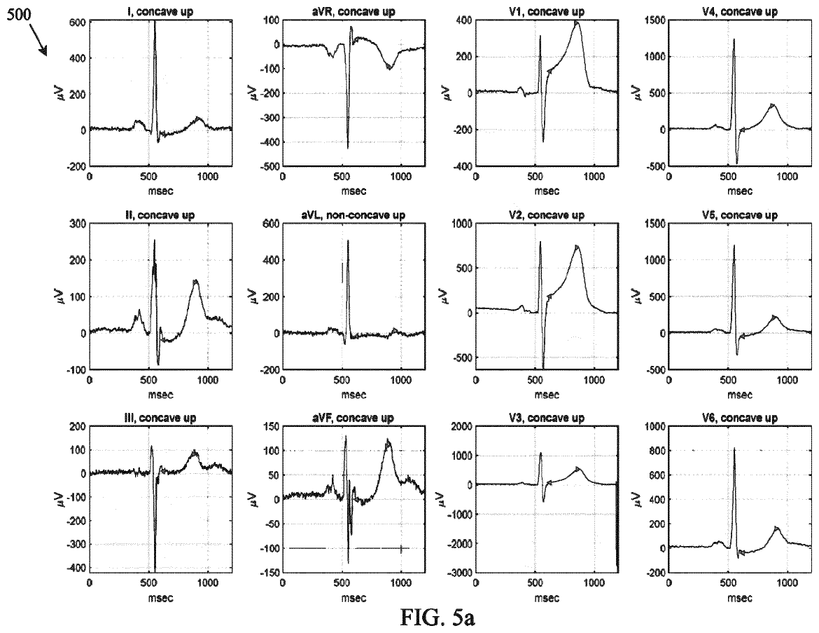

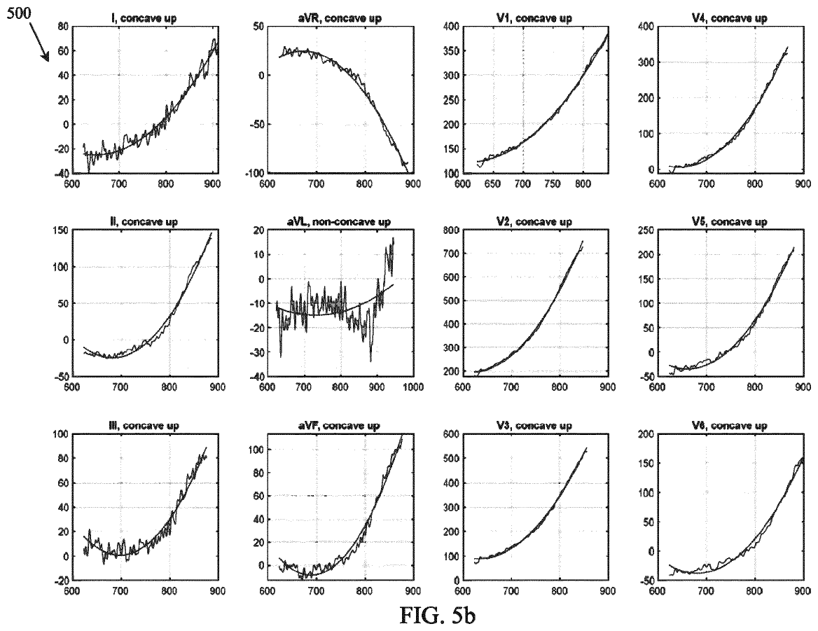

FIG. 5a shows a first set 500 of 12-lead ECG representative beats and FIG. 5b shows the magnified JTpeak intervals for the representative beats. FIGS. 5a-5b are representative of a patient having pericarditis. Each of the ECGs is labeled with the lead name (i.e., I, II, III, aVR, aVL, aVF, V1, V2, V3, V4, V5, or V6) and its upward concavity status. The start and end of the JTpeak intervals are marked on each representative beat. FIG. 5b shows a curve fitted to the beat. It is noted that lead aVR has a reverse direction from the other leads. Thus, an upward or downward concavity determination for the aVR lead is reversed to match the concavity definition for other leads.

As may be seen in FIG. 5b, each of the leads showing ST-elevation have upward concave ST segments only. Lead aVL does not have an upward concave ST segment, however, lead aVL also does not show ST-elevation. The diagnostic criteria for determining pericarditis include a threshold ST elevation that may be, e.g. 50 uV, although different guidelines provide different definitions. Thus, the system 100, when presented with this ECG data 500, would determine that the criteria for pericarditis are met and that the condition is not STEMI.

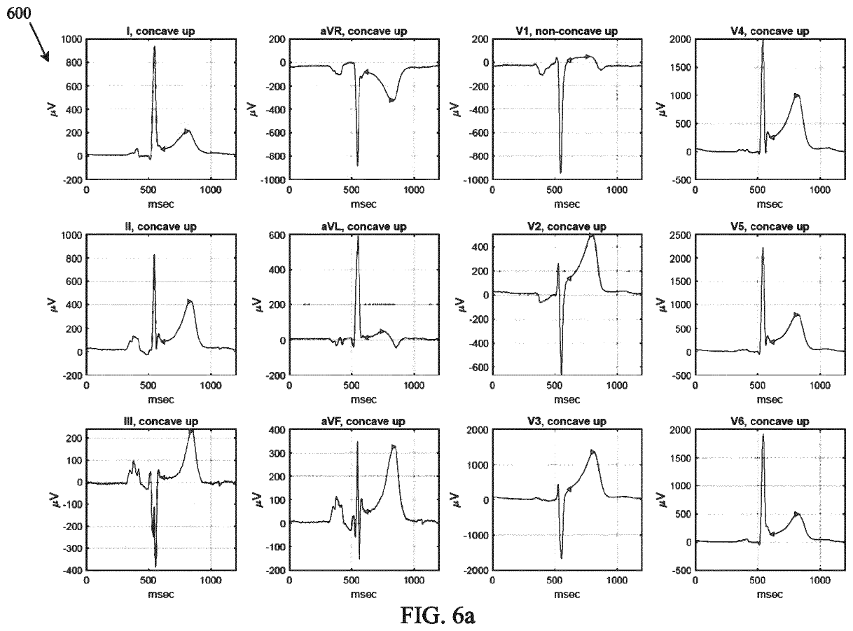

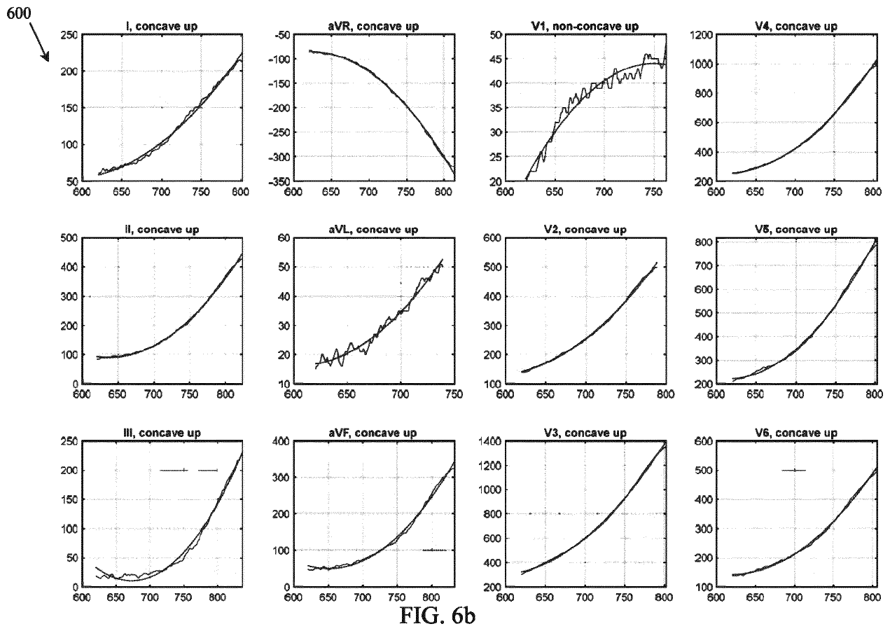

FIG. 6a shows a second set 600 of 12-lead ECG representative beats and FIG. 6b shows the magnified JTpeak intervals for the representative beats. FIGS. 6a-6b are representative of a patient having early repolarization. As may be seen in FIG. 6b, each of the leads showing ST elevation have upward concave ST segments only. Lead V1 does not have an upward concave ST segment, however, lead V1 also does not show ST-elevation. Similar to the pericarditis criteria, the diagnostic criteria for determining early repolarization also include a threshold ST elevation that may be, e.g. 100 uV. Thus, the system 100, when presented with this ECG data 600, would determine that the criteria for early repolarization are met and that the condition is not STEMI.

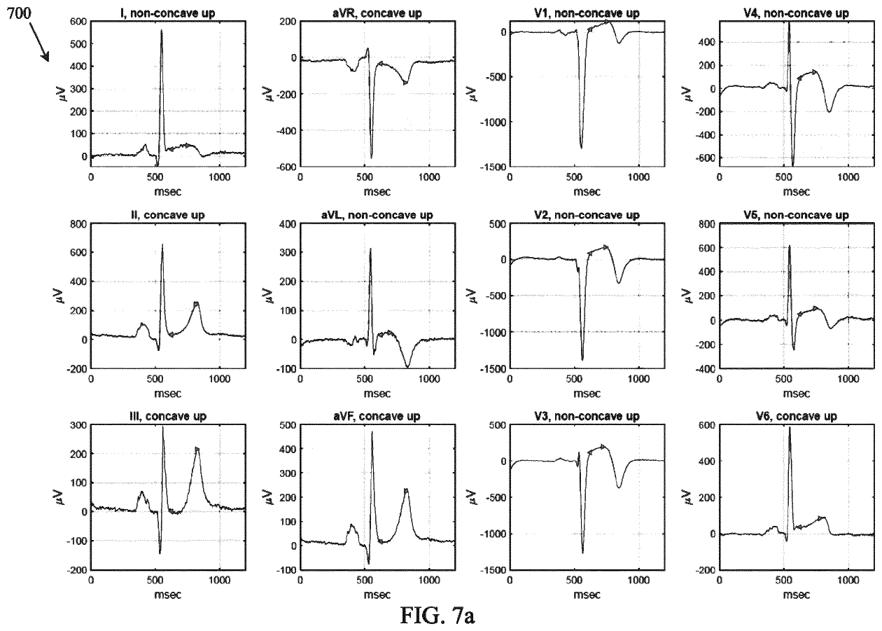

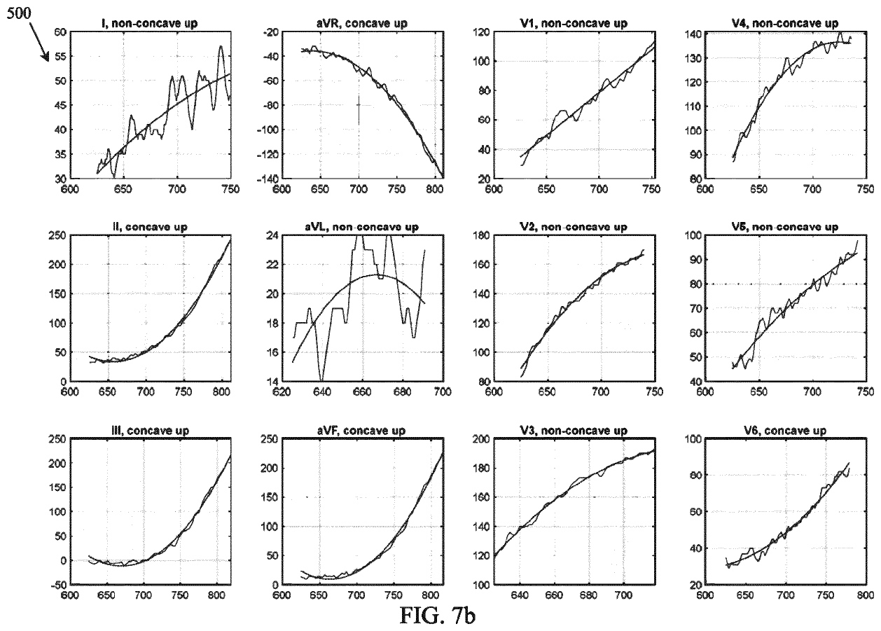

FIG. 7a shows a third set 700 of 12-lead ECG representative beats and FIG. 7b shows the magnified JTpeak intervals for the representative beats. FIGS. 7a-7b are representative of a patient having STEMI. As may be seen in FIG. 7b, multiple leads showing ST elevation (e.g. leads V2, V3, V4) have non-concave upward (concave downward, straight line, or too noisy to determine) segments. Similar to the previously discussed conditions, the diagnostic criteria for determining STEMI also include a threshold ST elevation that may be, e.g. 100 uV. Thus, the system 100, when presented with this ECG data 700, would determine that the criteria for STEMI are met and that the condition is STEMI.

The exemplary graphs shown in FIGS. 5-7 may be calculated and analyzed without visualization of the curves and extracted features. In such a situation, the system 100 may present its condition determination on the display 106 in e.g. textual form. However, it may be desirable for the user interpreting the results to consider both the automatic determination and the elements going into the determination, i.e., the fitted curve and the extracted features, by visualizing the curve and the associated features.

In 240, the fitted JTpeak interval curve and/or osculating circle and/or extracted features of the curve may be visually presented on the display 106.

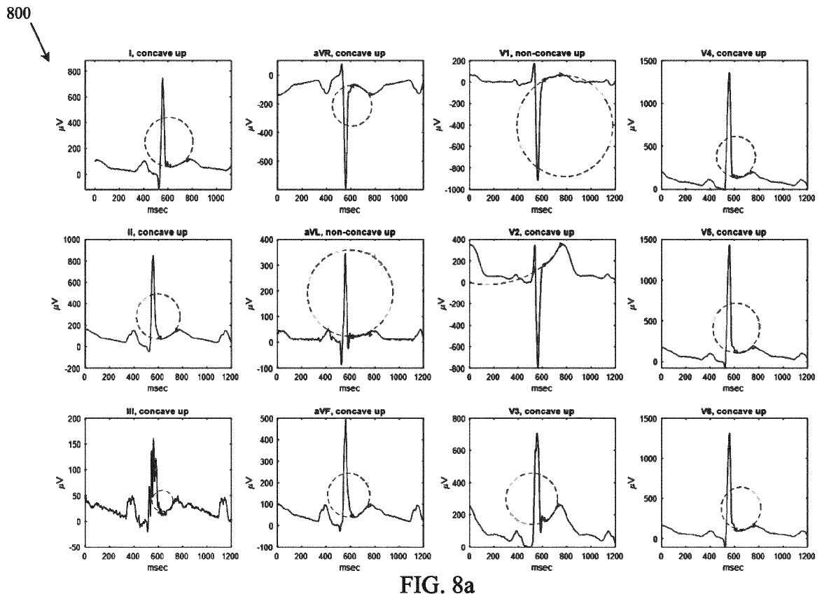

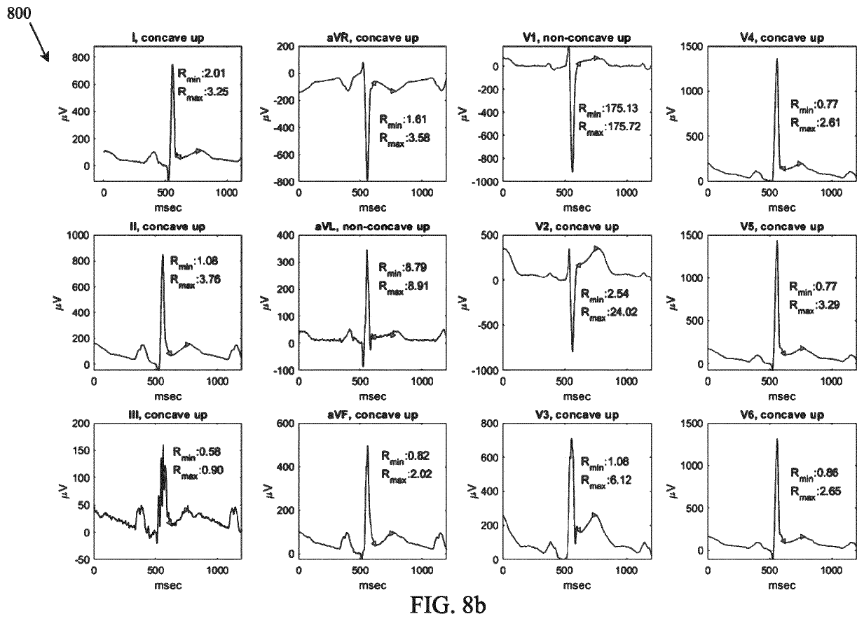

FIG. 8a shows a fourth set 800 of 12-lead ECG representative beats with an osculating circle visualized thereon. The osculating circle may be drawn at various points on the curve. For example, an osculating circle near the midpoint of the JTpeak interval may be desirable, or at either of the ends of the JTpeak interval. FIG. 8b shows the fourth set 800 of beats with minimum curvature radius and maximum curvature radius printed thereon.

The aforementioned visualization is only exemplary, and the aspects of the visualization may be modified by a user. For example, the best-fit curve may be shown, or different features of the curve may be printed.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method, comprising:
identifying a segment in ECG data;
modeling the segment with a curve;
extracting features from the curve;
determining a concavity of the segment based on the extracted features; and
determining a cardiological condition based on the concavity of the segment.

2. The method of claim 1, wherein the segment is a JTpeak interval, the JTpeak interval beginning at a J-point and ending at an apex of a T wave.

3. The method of claim 2, wherein the ECG data comprises a graph for each of twelve ECG leads and the concavity for each segment is determined.

4. The method of claim 3, wherein the cardiological condition is determined based on the concavity for at least one of the segments being non-concave upward.

5. The method of claim 1, wherein the curve is a second order polynomial and the extracted features comprise a model error, a noise level of the ECG data as compared with the curve, a vertex location and a maximum curvature.

6. The method of claim 5, wherein each of the extracted features are compared to predefined thresholds and, when the threshold conditions are met, the segment is determined to be concave upward.

7. The method of claim 5, further comprising:
generating an osculating circle for a point on the curve; and
visualizing the second order polynomial or the osculating circle overlaid on the ECG data.

8. The method of claim 5, further comprising:
visualizing one or more features overlaid on the ECG data.

9. The method of claim 1, wherein the cardiological condition is STEMI, the STEMI condition being distinguished from other cardiological conditions showing ST-elevation.

10. A computer readable storage medium comprising a computer program that when executed by a processor, performs the method of claim 1.

11. A system, comprising:
a memory configured to store ECG data; and
a processor configured to perform operations comprising:
identifying a segment in the ECG data;
modeling the segment with a curve;
extracting features from the curve;
determining a concavity of the segment based on the extracted features; and
determining a cardiological condition based on the concavity of the segment.

12. The system of claim 11, wherein the segment is a JTpeak interval, the JTpeak interval beginning at a J-point and ending at an apex of a T wave.

13. The system of claim 12, further comprising:
an ECG arrangement configured to generate the ECG data, wherein the ECG data comprises a graph for each of twelve ECG leads and the concavity for each segment is determined.

14. The system of claim 13, wherein the cardiological condition is determined based on the concavity for at least one of the segments being non-concave upward.

15. The system of claim 11, wherein the curve is a second order polynomial and the extracted features comprise a model error, a noise level of the ECG data as compared with the curve, a vertex location and a maximum curvature.

16. The system of claim 15, wherein each of the extracted features are compared to predefined thresholds and, when the threshold conditions are met, the segment is determined to be concave upward.

17. The system of claim 15, wherein the processor is configured to perform further operations comprising:
generating an osculating circle for a point on the curve.

18. The system of claim 17, further comprising:
a display configured to visualize the second order polynomial or the osculating circle overlaid on the ECG data.

19. The system of claim 18, wherein the display is further configured to:
visualize one or more features overlaid on the ECG data.

20. The system of claim 11, wherein the cardiological condition is STEMI, the STEMI condition being distinguished from other cardiological conditions showing ST-elevation.
